(19) United States
(12) Patent Application Publication
(10) Pub. No.: US 2003/0032099 A1
(43) Pub. Date: Feb. 13, 2003

(54) Methods for predicting susceptibility to obesity and obesity-associated health problems

(76) Inventors: Thomas J. Hudson, Montreal (CA); James C. Engert, Montreal (CA); Daniel Gaudet, Chicoutimi (CA); Pierre Lepage, Montreal (CA); Marie-Claude Vohl, Cap-Rouge (CA); Scott M. Williams, Nashville, TN (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/117,983

(22) Filed: Apr. 4, 2002

Related U.S. Application Data
(60) Provisional application No. 60/281,449, filed on Apr. 4, 2001.

(51) Int. Cl. C12P 21/06

(52) U.S. Cl. 435/69.1

(57) ABSTRACT

Methods for determining an individual's susceptibility to obesity or to a health disorder associated with obesity, comprising detecting an allele at a polymorphic site genetically linked to the resistin gene locus, wherein the allele is further linked to obesity, and wherein detection of the allele is indicative of the patient's susceptibility to a health disorder associated with obesity are provided. Also included in the present disclosure are nucleic acid molecules containing allelic variants at polymorphic sites.

### RELATED APPLICATION

This application claims the benefit of U.S. Provisional Application No. 60/281,449, filed Apr. 4, 2001, the entire teachings of which are incorporated herein by reference.

### GOVERNMENT SUPPORT

The invention was supported, in whole or in part, by grant numbers F32HL10457-01 and K14HL03321 from the National Institutes of Health. The Government has certain rights in the invention.

### BACKGROUND OF THE INVENTION

The frequency of obesity is increasing worldwide. Obesity is associated with health disorders such as diabetes mellitus, coronary heart disease, cancer, and sleep-breathing disorders. Indeed, the recent increase in numbers of obese people is cited as a reason for the corresponding rise in the number of cases of Type II diabetes. The documented association of obesity with disorders such as diabetes suggests a need for a better understanding of both the factors that affect obesity and the mechanisms by which obesity is related to other disorders.

Notions of obese individuals as lacking the will power sufficient to curb eating habits are being replaced by theories that obesity has specific genetic and molecular determinants. From genetic evidence, it is becoming clear that factors leading to a predisposition for obesity are heritable, thus implying the existence of one or more genes responsible for causing obesity. Identification of these genes and characterization of their gene products will provide clues to the treatment of obesity and disorders commonly associated with obesity.

### SUMMARY OF THE INVENTION

Diabetes and obesity have long been known to be related. The adipocyte hormone resistin (also called FIZZ3/ADSF) has been implicated as a molecular link between impaired glucose tolerance and obesity in mice. A search for sequence variants at the human resistin locus described herein identified nine single-nucleotide polymorphisms (SNPs), but no coding sequence variants. An investigation of the association of these SNPs with diabetes and obesity revealed two 5′ flanking variants in strong linkage disequilibrium that are associated with elevated body mass index (BMI). This investigation, combined with additional searches for other phenotypes commonly associated with diabetes, led to the identification of a total of nine SNPs that are described herein. The SNPs identified and described herein are useful for diagnostic, therapeutic and drug discovery methods associated with obesity and obesity-related disorders, diabetes mellitus, coronary heart disease, cancer and sleep-breathing disorders.

In one embodiment, the invention is directed to a method for determining an individual's susceptibility to obesity, comprising detecting in a biological sample obtained from the individual an allele at a polymorphic site genetically linked to the resistin gene locus, wherein the allele is further linked to obesity, and wherein detection of the allele is indicative of the patient's susceptibility to obesity. In a particular embodiment, the individual is determined to be heterozygous or homozygous for the allele. In one embodiment, susceptibility to obesity indicates a risk for an obesity-associated health disorder. The obesity-associated health disorder can be diabetes mellitus, coronary heart disease, obesity, cancer or sleep-breathing disorders.

In one embodiment, the invention is directed to a method for determining an individual's susceptibility to obesity, comprising detecting in a biological sample obtained from the individual an allele at a polymorphic site selected from the group consisting of: g.-638, g.-537, g.-420, g.-358, IVS2+39, IVS2+181, IVS3+30, IVS3−16, c.*62, and combinations thereof, and wherein detection of the allele is indicative of the individual's susceptibility to obesity. In a particular embodiment, the individual is of French-Canadian descent. In one embodiment, the polymorphic site is g.-537. In a different embodiment, the polymorphic site is g.-420. In a particular embodiment, the allele detected is selected from the group consisting of: g.-638G>A, g.-537A>C, g.-420C>G, g.-358G>A, IVS2+39C>T, IVS2+181G>A, IVS3+30C>T, IVS3−16C>G, and c.62*G>A. In a particular embodiment, the allele detected is g.-537A>C. In another embodiment, the allele detected is g.-420C>G. In one embodiment, the method of detecting the allele comprises a hybridization assay and/or use of a microarray.

In another embodiment, the invention is directed to a method for determining an individual's susceptibility to obesity or obesity-related health disorders, comprising detecting in a biological sample obtained from the individual an allele at a polymorphic site in the coding sequence of the resistin gene, wherein the allele is further linked to obesity, and wherein detection of the allele is indicative of the individual's susceptibility to obesity. In a particular embodiment, detecting the allele comprises the use of antibodies. In one embodiment, the allele is c.62*G>A.

In another embodiment, the invention is directed to a method for determining an individual's predisposition to insulin resistance and health disorders associated with insulin resistance, comprising detecting a specific allele at a polymorphic site, wherein the allele is genetically linked to the resistin gene locus, and wherein detection of the allele is indicative of insulin resistance.

In another embodiment, the invention is directed to a method for determining an individual's predisposition to insulin resistance and/or health disorders associated with insulin resistance, comprising detecting a specific allele at a polymorphic site, wherein the allele is selected from the group consisting of: g.-638G>A, g.-537A>C, g.-420C>G, g.-358G>A, IVS2+39C>T, IVS2+181G>A, IVS3+30C>T, IVS3−16C>G, and c.62*G>A, and wherein detection of the allele is indicative of the individual's susceptibility to insulin resistance and/or health disorders associated with insulin resistance.

In yet another embodiment, the invention is directed to a method for determining an individual's predisposition to a health disorder associated with insulin resistance, comprising detecting a specific allele at a polymorphic site, wherein the allele is genetically linked to the resistin gene locus, and wherein detection of the allele is indicative of the health disorder associated with insulin resistance.

In another embodiment, the invention is directed to a method for determining an individual's predisposition to a health disorder associated with insulin resistance, comprising detecting a specific allele at a polymorphic site, wherein the allele is selected from the group consisting of: g.-638G>A, g.-537A>C, g.-420C>G, g.-358G>A, IVS2+39C>T, IVS2+181G>A, IVS3+30C>T, IVS3−16C>G and c.62*G>A, and wherein detection of the allele is indicative of the individual's susceptibility to a health disorder associated with insulin resistance.

In another embodiment, the invention is directed to a method for determining an individual's predisposition to type II diabetes, comprising detecting a specific allele at a polymorphic site, wherein the allele is genetically linked to the resistin gene locus, and wherein detection of the allele is indicative of type II diabetes.

In another embodiment, the invention is directed to a method for determining an individual's predisposition to type II diabetes, comprising detecting a specific allele at a polymorphic site, wherein the allele is selected from the group consisting of: g.-638G>A, g.-537A>C, g.-420C>G, g.-358G>A, IVS2+39C>T, IVS2+181G>A, IVS3+30C>T, IVS3−16C>G, and c.62*G>A, and wherein detection of the allele is indicative of the individual's susceptibility to type II diabetes.

In yet another embodiment, the invention is directed to a nucleic acid molecule comprising a nucleotide sequence from the resistin gene comprising an allele selected from the group consisting of: g.-638G>A, g.-537A>C, g.-420C>G, g.-358G>A, IVS2+181G>A, IVS3+30C>T and IVS3−16C>G, wherein the sequence flanks the allele on one or both sides.

### BRIEF DESCRIPTION OF THE DRAWINGS

FIG. 1 is a schematic representation of the human resistin (FIZZ3) gene locus and exon structure and location of nine SNPs. Minor allele frequencies derived from all Quebec samples assayed are in parentheses (based on >300 samples for all SNPs except IVS3+30 which is based on 48 samples). SNP numbering is based on GenBank accession number AF205952.

FIG. 2A and FIG. 2B are graphical representations of average BMI by genotype for g.-420 (A) and g.-537 (B). In FIG. 2A, closed circles represent the CC genotype, and open circles represent CG and GG genotypes combined. In FIG. 2B, closed circles represent the AA genotype, and open circles represent AC and CC genotypes combined. Error bars show the S.E. of the mean. p-values from Student's t test are shown above each comparison. DBT: diabetic; SLSJ: Saguenay-Lac-St-Jean; QC: Quebec City; All Non-DBT: all non-diabetics from SLSJ combined with the Quebec City samples.

FIG. 3 is a table showing the genotype frequencies of the 5′ flanking SNPs at the resistin locus. Percentage of each genotype is shown in parentheses. SLSJ: Saguenay-Lac-St-Jean.

FIG. 4 is a table showing subjects' characteristics by genotype in the Quebec city sample of non-diabetic men. Data shown are mean±standard deviation. The number of subjects is shown in parentheses.

FIG. 5 is a table showing the logistic regression analyses for BMI>30 for 5′ flanking SNPs at the resistin locus.

### DETAILED DESCRIPTION OF THE INVENTION

Approximately 80% of type II diabetics are overweight or obese. The growing rate of obesity is directly related to the increasing prevalence of type II diabetes in North America. The present invention makes use of a molecular link between insulin resistance, a phenotype common to diabetics, and obesity. The molecular link is the product of the resistin gene, which has been implicated in causing insulin resistance (Steppan, C. et al., Nature. 2001, 409:307-312). As described herein, particular sequence variants at the resistin gene locus have been linked to obesity and obesity-related health problems.

The gene encoding resistin hormone (gene accession #NM_{—}020415) has been postulated to play a role at the nexus of type II diabetes and obesity (Steppan, C. et al., 2001. Nature, 409:307-312). Resistin belongs to a family of secreted peptides (Holcomb I. et al., 2000. EMBO J., 19:4046-55; Steppan C. et al., 2001. Proc. Natl. Acad. Sci. USA, 409:307-312) that shares an invariant cysteine-rich C-terminus and forms disulfide linked homodimers (Banerjee R. et al., 2001. J. Biol. Chem., 276:25970-3). Resistin was identified in the mouse by screening differentiated adipocytes for genes repressed by the anti-diabetic drug rosiglitazone. Rosiglitazone is a member of the class of insulin sensitizing drugs known as thiazolidinediones (TZDs) whose exact mode of action is unknown but are thought to target the nuclear receptor peroxisome proliferator-activated receptor, gamma (PPAR-γ; Spiegelman, B. et al., 1988. Diabetes, 47:507-14). Mouse resistin is expressed exclusively in adipocytes and appears to inhibit the differentiation of adipocytes in culture (Steppan, C. et al., 2001. Nature, 409:307-312; Kim K. et al., 2001. J. Biol. Chem., 276:11252-11256). Circulating levels of resistin are increased after a high carbohydrate meal, as well as in the presence of both genetically and diet-induced obesity (Steppan, C. et al., 2001. Nature, 409:307-312; Kim K. et al., 2001. J. Biol. Chem., 276:11252-11256). In mice fed a high-fat diet, blood sugar and insulin action are improved by the administration of an anti-resistin antibody, possibly through the regulation of glucose transport (Steppan, C. et al., 2001. Nature, 409:307-312).

The present invention relates to methods and compositions for characterization of “alleles” that are in “linkage disequilibrium” with a particular version of a gene known to be associated with “insulin resistance,” i.e., reduced biological response to either exogenous or endogenous insulin. As used herein, “allele” refers to a specific sequence variant possible at a polymorphic site. A “polymorphic site” is a position in a polynucleotide sequence that can have more than one possible allele. “Polymorphic” is a comparative term that compares a sequence to at least one other sequence. For example, at a particular site on a chromosome or in a reference sequence, one individual in a population might have a guanine while another individual might have an adenine. Such a site is a polymorphic site having two different alleles at that site; one allele has a guanine at the polymorphic site, while the other allele has an adenine at the polymorphic site. Any sequence position can be referred to as a polymorphic site provided more than one possible allele occurs at the site.

As used herein, “linkage disequilibrium” relates heritable elements (e.g., alleles, phenotypes, genotypes) such that there is a tendency that specific combinations of elements are inherited together instead of inherited independently by random assortment, e.g., a specific allele of a gene can be said to be in linkage disequilibrium with a specific phenotype if, in a population, the genotype of an individual displaying the phenotype is more likely to carry the particular allele than would be expected if the allele and phenotype were inherited independently of each other. Alleles are randomly assorted or inherited independently if the frequency of the two alleles together is the product of the frequencies of the two alleles individually. For example, if two alleles at different polymorphic sites are present in 50% of the chromosomes in a population, then they would be said to assort randomly if the two alleles are present together on 25% of the chromosomes in the population. A higher percentage would mean that the two alleles are linked. For example, a polymorphic site, “g.-50” (see below for an explanation of nomenclature), having two alleles, “g.-50A” and “g.-50C”—each appearing in 50% of the individuals in a given population, is said to be in linkage disequilibrium with respect to another polymorphic site, “g.-75,” having two alleles, “g.-75G” and “g.-75T”—each appearing in 50% of the individuals in a given population, if particular combinations of alleles (e.g., g.-50A/g.-75G) are observed in individuals at a frequency greater than 25% (if the polymorphic sites are not linked, then one would expect a 50% chance of an individual having g.-50A and a 50% chance of having g.-75G—thus leading to a 25% chance of having the combination of g.-50A/g.-75G together). Heritable elements that are in linkage disequilibrium are said to be “linked” or “genetically linked” to each other.

A systematic nomenclature has been proposed for describing polymorphic sites and alleles. Sequence variations are described in relation to a reference sequence, e.g., sequences referenced by database accession numbers. The first letter (followed by a period), denotes the source of the sequence, e.g., “g.” denotes a genomic sequence, “c.” denotes a cDNA sequence, “m.” denotes a mitochondrial sequence, “r.” denotes an RNA sequence, and “p.” denotes a protein sequence. Following the source, a number corresponding to the first affected nucleotide in the reference sequence is followed by the reference nucleotide, the “>” symbol which denotes a substitution, and the variant nucleotide. For example, an adenine at position 76 in the reference sequence that has a variant thymidine at the position in the sequence to be named, would be referred to as “g.76A>T” when in reference to a genomic sequence. According to convention, nucleotide “+1” is the adenine of the ATG start codon; the nucleotide 5′ to +1 is numbered “−1”. There is no nucleotide corresponding to “0”. The nucleotide 3′ of the translation termination codon is “* 1”. Intron sequences are designated by the nucleotide number corresponding to the last nucleotide of the preceding exon, a “+”, and the position in the intron of the affected nucleotide. For example, a G>T variant 35 nucleotides 3′ of the end of exon 1 (which occurs at nucleotide number 75 of the reference sequence), is designated, “g.75+35G>T”. Alternatively, if the exon number is known, the variant can be described as “g.IVS1+35G>T” since the variant position occurs 3′ from exon 1.

In particular, the present invention relates to, but is not limited to, single nucleotide polymorphisms (hereinafter, “SNP,” is used to refer to a polymorphic site that is a single nucleotide as opposed to several nucleotides in length, or, when a reference sequence is known, “SNP” can be used to refer to a specific allele at a single nucleotide polymorphic site), that are located near the resistin gene locus; the product of the resistin gene has been implicated in insulin resistance (Steppan, C. et al., Nature. 2001. 409:307-312). These SNPs are in linkage disequilibrium with obesity, as measured by quantifiable indices such as, e.g., “body mass index” (hereinafter, “BMI;” the ratio of a person's mass in kilograms to the square of the person's height in meters). An elevated BMI of >30 kg/m² is an indicator of obesity. In other words, a particular allele at a polymorphic site described herein is associated with obesity. As such, the SNPs described herein are used as “genetic markers,” i.e., sequence elements that are indicative of other sequence elements or phenotypes, e.g., insulin resistance, obesity or diabetes. The methods of the present invention are not limited to the use of SNPs as genetic markers, as other alleles representing larger polymorphic sites (e.g., substitutions, deletions, insertions or translocations that span more than a single nucleotide) can serve as genetic markers for insulin resistance or disorders associated with obesity.

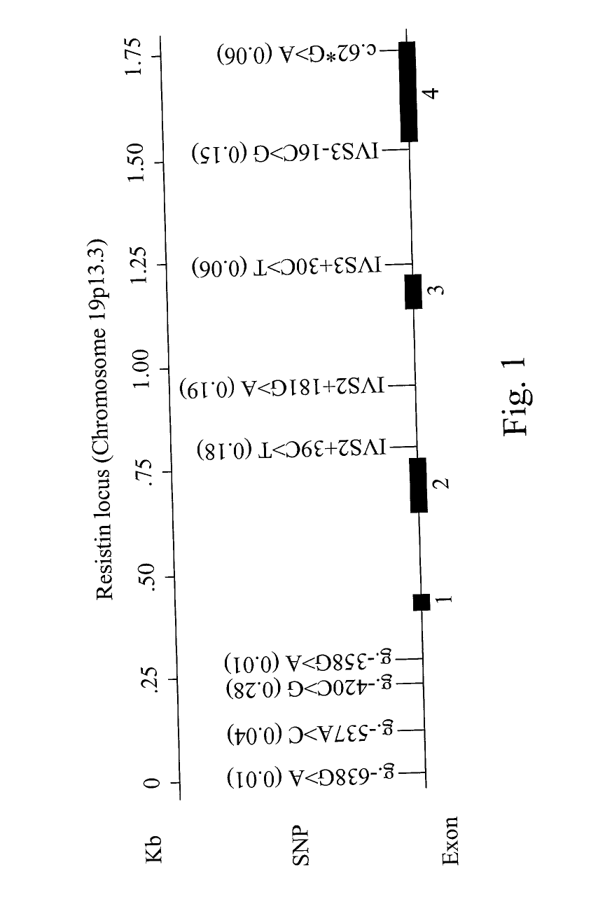

A search for sequence variants in and around the resistin gene locus (FIG. 1) led to the identification of nine SNPs. Two alleles in the 5′ region flanking the resistin gene, in linkage disequilibrium with each other, are linked to an elevated BMI in individuals from Quebec; elevated BMI is an indicator of obesity. The resistin hormone is a mediator of insulin resistance (Steppan, C. et al., Nature. 2001. 409:307-312) and has been postulated to play a role at the nexus of obesity and diabetes (Steppan, C. et al., Nature. 2001. 409:307-312). Resistin is a circulating protein, secreted specifically by adipocytes, that antagonizes insulin action. Resistin is a member of a secreted peptide family (Holcomb, I. et al., EMBO J. 2000. 19:4046-4055; Steppan, C. et al., Proc. Natl. Acad. Sci. USA. 2001. 98:502-506) that was cloned in mouse by screening differentiated adipocytes for genes repressed by the anti-diabetic drug, rosiglitazone, a member of the class of drugs known as thiazolidinediones that target the PPARγ nuclear receptor (Spiegelman, B. Diabetes. 1998. 47:507-514).

In addition to their physical proximity to the resistin gene locus, the SNPs described herein are linked to phenotypic indicators of obesity, namely, elevated body weight, BMI, body fat mass, waist circumference and abdominal subcutaneous adipose tissue area measured by computed tomography. Obesity is associated with other health problems such as diabetes, coronary heart disease, certain forms of cancer and sleep-breathing disorders. The proximity of the SNPs to the resistin gene locus indicates that the SNPs described herein are useful as markers for an obese phenotype and as markers for particular versions of the resistin gene. As the product of the resistin gene appears to play a role in the association of obesity and type II diabetes, the SNPs described herein are useful as markers or potential therapeutic targets for any disorders associated with insulin resistance or disorders genetically linked to the specific SNPs or the resistin gene locus.

Insulin resistance is a hallmark of Type II diabetes. Diabetes mellitus is characterized by the inability or impaired ability of the body to clear glucose from the blood. Glucose clearance is controlled by the hormone, insulin. For Type I diabetes, an auto-immune disorder, insulin levels are not maintained at sufficient levels to clear glucose. For Type II diabetes, however, insulin levels are sufficiently high, but the insulin signal is not properly relayed in order to effect glucose clearance. The inability of insulin to signal is referred to as “insulin resistance.” Type II diabetes is associated with obesity and is characterized by insulin resistance.

In addition to type II diabetes, obesity is associated with coronary heart disease, certain forms of cancer and sleep-breathing disorders (Kopelman, P. Nature. 2000. 404:635-643). The SNPs described herein can be used as genetic markers or therapeutic targets from these disorders. In addition to the known disorders associated with obesity, the SNPs described herein are useful markers and potential therapeutic targets for other disorders not yet demonstrated to be associated with obesity.

In general, the detection in a biological sample obtained from an individual of a particular allele at a polymorphic site that is genetically linked to a particular gene or phenotype, is indicative of a particular allele of the gene or of the presence of the particular phenotype. It is the genetic linkage of a particular allele to a particular phenotype that allows for the inference to be made that, the detection of the allele in a sample indicates that the individual from whom the sample was obtained also exhibits the linked phenotype. A phenotype can be, for example, obesity, a predisposition to obesity, obesity-related diseases and health disorders, or a characteristic of such diseases such as, for example, insulin resistance. The sample to be assessed can be any sample that contains a gene expression product. Suitable sources of gene expression products, i.e., samples, can include cells, lysed cells, cellular material for determining gene expression, or material containing gene expression products. Examples of such samples are blood, plasma, lymph, urine, tissue, mucus, sputum, saliva or other cell samples. Methods of obtaining such samples are known in the art. For the purposes of the invention, individuals from whom samples are obtained can be, for example, human patients. Such patients may or may not exhibit obese characteristics or phenotypes or diseases related to obesity. Additionally, samples can be obtained from humans that are undergoing treatment for obesity or obesity-related diseases.

As used herein, “gene expression products” are proteins, polypeptides, or nucleic acid molecules (e.g., mRNA, tRNA, rRNA, cDNA, or cRNA) that result from transcription and/or translation of genes. The nucleic acid molecule levels measured can be derived directly from the gene or, alternatively, from a corresponding regulatory gene or regulatory sequence element. All forms of gene expression products can be measured. For example, the nucleic acid molecule can be transcribed to obtain an RNA gene expression product. If desired, the transcript can be translated using, for example, standard in vitro translation methods to obtain a polypeptide gene expression product. Polypeptide gene expression products can be detected in protein binding assays, for example, antibody assays, or in nucleic acid binding assays, known in the art. Additionally, variants of genes and gene expression products including, for example, spliced variants and polymorphic alleles, can be measured. Similarly, gene expression can be measured by assessing the level of a polypeptide or protein or derivative thereof translated from mRNA. The sample to be assessed can be any sample that contains a gene expression product. Suitable sources of gene expression products, e.g., samples, can include intact cells, lysed cells, cellular material for determining gene expression, or material containing gene expression products. Examples of such samples are intestinal tissue, cells derived from intestinal tissue, nucleic acids or polypeptides derived from intestinal tissue, blood, plasma, lymph, urine, tissue, mucus, sputum, saliva, or other cell samples. Methods of obtaining such samples are known in the art.

Methods are well known in the art for detection of alleles at specific polymorphic sites, including sequencing, PCR-based assays and hybridization assays. If the site is in linkage disequilibrium with a particular phenotype, i.e., obesity, then the detection of a specific allele is indicative of the particular phenotype. Thus, diagnostic tests can be performed quickly and accurately for any phenotypes that are genetically linked to alleles at the polymorphic sites described herein.

The invention relates to detection of alleles at polymorphic sites. The detection methods can include hybridization assays that detect DNA fragments that include the polymorphic site and portions of complements of the alleles that encompass the polymorphic site. Such fragments are at least 5, and preferably at least 10, nucleotides in length. Such fragments including the polymorphic site can be, for example, 5-10, 5-15, 10-20, 5-25, 10-30, 10-50 or 10-100 bases in length. The additional nucleotides can be on one or both sides of the polymorphic site.

The invention further provides allele-specific oligonucleotides (e.g., probes and primers) that hybridize to one or more allelic variants described in FIG. 1, or to their complementary sequences. Such oligonucleotides will hybridize to one polymorphic form of the nucleic acid molecules described herein but not to the other polymorphic form(s) of the sequence, i.e., are allele-specific. Thus, such oligonucleotides can be used to determine the presence or absence of particular alleles of the polymorphic sequences described herein.

Hybridization probes are oligonucleotides that bind in a base-specific manner to a complementary strand of nucleic acid. Such probes include peptide nucleic acids (hereinafter, “PNA”), as described in Nielsen et al. (1991. Science 254, 1497-1500). Probes can be any length suitable for specific hybridization to the target nucleic acid sequence. The most appropriate length of the probe may vary depending upon the hybridization method in which it is being used; for example, particular lengths may be more appropriate for use in microfabricated arrays (microarrays), while other lengths may be more suitable for use in classical hybridization methods. Such optimizations are known to the skilled artisan. Suitable probes and primers can range from about 5 nucleotides to about 30 nucleotides in length. For example, probes and primers can be 5, 6, 8, 10, 12, 14, 16, 18, 20, 22, 24, 25, 26, 28 or 30 nucleotides in length. Additionally, a probe can be a genomic fragment that can range in size from about 25 to about 2,500 nucleotides in length. The probe or primer preferably overlaps at least one polymorphic site occupied by any of the possible variant nucleotides. The nucleotide sequence can correspond to the coding sequence of the allele or to the complement of the coding sequence of the allele.

Hybridizations can be performed under stringent conditions, e.g., at a salt concentration of no more than 1 M and a temperature of at least 25° C. For example, conditions of 5×SSPE (750 mM NaCl, 50 mM Na-Phosphate, 5 mM EDTA, pH 7.4) and a temperature of 25-30° C., or equivalent conditions, are suitable for allele-specific probe hybridizations. Equivalent conditions can be determined by varying one or more of the parameters given as an example, as known in the art, while maintaining a similar degree of identity or similarity between the target nucleotide sequence and the primer or probe used.

Conditions for stringency can be as described in WO 98/40404, the teachings of which are incorporated herein by reference. In particular, examples of “highly stringent,” “stringent,” “reduced,” and “least stringent” conditions are provided in WO 98/40404 in the Table on page 36, which is reproduced below. Highly stringent conditions are those that are at least as stringent as, for example, conditions A-F; stringent conditions are at least as stringent as, for example, conditions G-L; and reduced stringency conditions are at least as stringent as, for example, conditions M-R. For the purposes of the present invention, detection of SNPs will typically utilize highly stringent hybridization and wash conditions.

| Strin- | | | | |
|---|---|---|---|---|
| gency | Poly- | Hybrid | Hybridization | Wash |
| Condi- | nucleotide | Length | Temperature | Temperature and |
| tion | Hybrid | (bp)^{‡} | and Buffer^{†} | Buffer^{†} |
| A | DNA:DNA | ≧50 | 65° C.; 1xSSC -or- | 65° C.; 0.3xSSC |
| | | | 42° C.; 1xSSC, | |
| | | | 50% formamide | |
| B | DNA:DNA | <50 | T_{B}*; 1xSSC | T_{B}*; 1xSSC |
| C | DNA:RNA | ≧50 | 67° C.; 1xSSC -or- | 67° C.; 0.3xSSC |
| | | | 45° C.; 1xSSC, | |
| | | | 50% formamide | |
| D | DNA:RNA | <50 | T_{D}*; 1xSSC | T_{D}*; 1xSSC |
| E | RNA:RNA | ≧50 | 70° C.; 1xSSC -or- | 70° C.; 0.3xSSC |
| | | | 50° C.; 1xSSC, | |
| | | | 50% formamide | |
| F | RNA:RNA | <50 | T_{F}*; 1xSSC | T_{F}*; 1xSSC |
| G | DNA:DNA | ≧50 | 65° C.; 4xSSC -or- | 65° C.; 1xSSC |
| | | | 42° C.; 4xSSC, | |
| | | | 50% formamide | |
| H | DNA:DNA | <50 | T_{H}*; 4xSSC | T_{H}*; 4xSSC |
| I | DNA:RNA | ≧50 | 67° C.; 4xSSC -or- | 67° C.; 1xSSC |
| | | | 45° C.; 4xSSC, | |
| | | | 50% formamide | |
| J | DNA:RNA | <50 | T_{J}*; 4xSSC | T_{J}*; 4xSSC |
| K | RNA:RNA | ≧50 | 70° C.; 4xSSC -or- | 67° C.; 1xSSC |
| | | | 50° C.; 4xSSC, | |
| | | | 50% formamide | |
| L | RNA:RNA | <50 | T_{L}*; 2xSSC | T_{L}*; 2xSSC |
| M | DNA:DNA | ≧50 | 50° C.; 4xSSC -or- | 50° C.; 2xSSC |
| | | | 40° C.; 6xSSC, | |
| | | | 50% formamide | |
| N | DNA:DNA | <50 | T_{N}*; 6xSSC | T_{N}*; 6xSSC |
| O | DNA:RNA | ≧50 | 55° C.; 4xSSC -or- | 55° C.; 2xSSC |
| | | | 42° C.; 6xSSC, | |
| | | | 50% formamide | |
| P | DNA:RNA | <50 | T_{P}*; 6xSSC | T_{P}*; 6xSSC |
| Q | RNA:RNA | ≧50 | 60° C.; 4xSSC -or- | 60° C.; 2xSSC |
| | | | 45° C.; 6xSSC, | |
| | | | 50% formamide | |
| R | RNA:RNA | <50 | T_{R}*; 4xSSC | T_{R}*; 4xSSC |
| ^{‡}The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{†}SSPE(1xSSPE is 0.15 M NaCl, 10 mM NaH₂PO₄, and 1.25 mM EDTA, pH 7.4) can be substituted for SSC (1xSSC is 0.15 M NaCl and 15 mM sodium citrate) in the hybridization and wash buffers; washes are performed for 15 minutes after hybridization is complete. | | | | |
| *T_{B}-T_{R}: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For | | | | |
| # hybrids between 18 and 49 base pairs in length, Tₘ(° C.) = 81.5 + 16.6(log₁₀[Na⁺]) + 0.41(% G + C) − (600/N), where N is the number of bases in the hybrid, and [Na⁺] is the concentration of sodium ions in the hybridization buffer ([Na⁺] for 1xSSC = 0.165 M). | | | | |

It will be clear to one of skill in the art that the contacting, hybridization and wash steps can be optimized using any suitable method of optimization established in the art. These include, but are not limited to, techniques that increase the efficiency of annealing or hybridization from complex mixtures of polynucleotides (e.g., PERT; Nucleic Acids Research 23:2339-2340, 1995) or hybridization in different formats (e.g., using an immobilized template or using microtiter plates; Analytical Biochemistry 227:201-209, 1995).

The polymorphisms can also be identified by hybridization to nucleic acid arrays, some examples of which are described in WO 95/11995. The same arrays or different arrays can be used for analysis of characterized polymorphisms. WO 95/11995 also describes subarrays that are optimized for detection of a variant form of a precharacterized polymorphism. Such a subarray contains probes designed to be complementary to a second reference sequence, which is an allelic variant of the first reference sequence. The second group of probes is designed by the same principles as described, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to 21 bases).

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W. H. Freeman and Co, New York, 1992), Chapter 7.

Alleles of target sequences can be differentiated using single-strand conformation polymorphism analysis, which identifies base differences by alteration in electrophoretic migration of single stranded PCR products (Orita et al., 1989. Proc. Nat. Acad. Sci. 86:2766-2770). Amplified PCR products can be generated as described above, and heated or otherwise denatured, to form single-stranded amplification products. Single-stranded nucleic acids may re-fold or form secondary structures that are partially dependent on the base sequence. The different electrophoretic mobilities of single-stranded amplification products can be related to base-sequence differences between alleles of target sequences.

An alternative method for identifying and analyzing polymorphisms is based on single-base extension (SBE) of a fluorescently-labeled primer coupled with fluorescence resonance energy transfer (FRET) between the label of the added base and the label of the primer. Typically, the method, such as that described by Chen et al., (PNAS 94:10756-61 (1997)), uses a locus-specific oligonucleotide primer labeled on the 5′ terminus with 5-carboxyfluorescein (FAM). This labeled primer is designed so that the 3′ end is immediately adjacent to the polymorphic site of interest. The labeled primer is hybridized to the locus, and single base extension of the labeled primer is performed with fluorescently-labeled dideoxyribonucleotides (ddNTPs). An increase in fluorescence of the added ddNTP in response to excitation at the wavelength of the labeled primer is used to infer the identity of the added nucleotide. Other suitable methods will be readily apparent to the skilled artisan.

The detection of a particular allele by any of the methods described herein in a sample derived from an individual is indicative of the individual's susceptibility to obesity, insulin resistance and/or type II diabetes mellitus. Particular alleles are linked to an increased probability of being susceptible to one or more of these disorders as compared with an appropriate control, and other alleles will be linked to a decreased probability of being susceptible to one or more of these disorders as compared with an appropriate control. For example, detection of either g.-537A>C, g.-420C>G in a sample obtained from an individual is indicative of the individual being highly susceptible to obesity. As the polymorphisms described herein are linked to the resistin gene locus, alleles described herein are also indicative of insulin resistance and diabetes mellitus, since it has been shown that the product of the resistin gene is important in effecting insulin resistance, which is a hallmark of diabetes mellitus.

In addition to methods of detecting SNPs, the invention is directed to using particular alleles as therapeutic targets for disorders associated with obesity in the cases where the particular allelic versions are functionally responsible for effecting the disease phenotype. For example, polymorphisms that have direct functional consequences on a gene product or the levels of a gene product can be the target of directed therapies to alleviate either the functional or regulatory consequences of the allele present at the particular polymorphic site.

The invention further provides kits comprising at least one allele-specific oligonucleotide or gene expression product indicator as described herein. Often, the kits contain one or more pairs of allele-specific oligonucleotides hybridizing to different forms of a polymorphism. In some kits, the allele-specific oligonucleotides are provided immobilized to a substrate. For example, the same substrate can comprise allele-specific oligonucleotide probes for detecting at least 10, 100 or all of the polymorphisms shown in the Table. Optional additional components of the kit include, for example, restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions. Usually, the kit also contains instructions for carrying out the methods.

The invention will be further described with reference to the following non-limiting examples. The teachings of all the patents, patent applications and all other publications and websites cited herein are incorporated by reference in their entirety.

### Exemplification

### EXAMPLE 1

### Research Design and Methods

Population samples. The diabetes case/control sample (n=359; mean age=52.0) has been described (Vohl M-C. et al., 2000. J. Lipid Res., 41:945-952). Briefly, cases were newly-diagnosed type II diabetics (defined by WHO98 criteria following a 75 g oral glucose load (Alberti, K. and Zimmet, P. 1998. Diabet. Med., 15: 539-553) from French-Canadian individuals from the Saguenay-Lac-St-Jean (SLSJ) region of Quebec, and age- and sex-matched controls (having a normal glucose tolerance). Some of these individuals were also enrolled in a family based study of 79 families comprising 424 individuals. The Quebec City area sample, comprised of 231 men (mean age=42.4) selected to cover a wide range of adiposity, has been previously described (Vohl—C. et al., 1999. Int. J. Obes. Relat. Meta. Disord., 23:918-925). Briefly, these subjects are sedentary, non-smoking and free of metabolic disorders requiring treatment such as diabetes, hypertension and coronary heart disease. The Scandinavian case/control sample (n=968; mean age=60.5) and the Scandinavian family-based study populations have been described (Altshuler, D. et al., 2000. Nat. Genet., 26:76-80). Briefly, cases had either overt diabetes or severe impaired glucose tolerance; cases and controls were matched for gender, age and geographic location. The Scandinavian family-based study populations, were based on trios in which the offspring had either type 2 diabetes (n=126) or impaired glucose tolerance (n=108) or impaired fasting glucose (n=99) or normal glucose tolerance (n=379). All patients gave informed consent and recruitment and research protocols were monitored by local Institutional Review Boards.

PCR amplification and sequencing. The annealing temperature for all primer pairs was 56-60° C. PCR conditions were as follows: 50 ng of template genomic DNA, 1.25 units Qiagen HotStart Taq polymerase (Qiagen, Valencia, Calif.) in the buffer recommended by the manufacturer, (1.5 mM MgCl₂,) 0.2 mM dNTPs, 0.4 mM primers all in a reaction volume of 50 mL. PCR products were purified with Millipore Mulitiscreen PCR 96 well plates (Millipore, Watertown, Mass.). Sequencing reactions were performed using BigDye Terminator Cycle sequencing ready reactions kit (version 2.0) (Applied Biosystems, Foster City, Calif.) and the products were analyzed on ABI 3700 automated DNA sequencers (Applied BioSystems, Foster City, Calif.). The run files were processed using Sequencing Analysis software (version 3.6) and then aligned and compared using Autoassembler 2.1 (Applied BioSystems, Foster City, Calif.). All four exons, the exon-intron splicing boundaries, as well as the promoter of the resistin gene were screened (see below for primers) by sequencing two CEPH individuals and 45 individuals from Quebec (SLSJ and Quebec City) consisting of obese and non-obese patients who were either non-diabetic or diagnosed with type 2 diabetes. All amplified fragments of the resistin gene exhibited the expected lengths, consistent with the absence of deletions, duplications or rearrangements within these fragments.

Primers used to sequence the resistin locus:

PromoterF tgtcattctc acccagagac a (SEQ ID No: 1)

PromoterR tgggctcagc taaccaaatc (SEQ ID No: 2)

Exon 1-2F gggacttatt agccaagcca (SEQ ID No: 3)

Exon 1-2R tgggttggag tcaggtctgt (SEQ ID No: 4)

Intron2F gagaggatcc aggaggtcg (SEQ ID No: 5)

Intron2R aggtgacgct ctggcact (SEQ ID No: 6)

Exon 3F acagggctag gggaggatg (SEQ ID No: 7)

Exon 3R agtagaggct ggacacggg (SEQ ID No: 8)

Exon 4F cctcagcctc ccagctca (SEQ ID No: 9)

Exon 4R agacgctaga tcagtccctc c (SEQ ID No: 10)

Statistical and analytical methods. Allele frequency and genotype distribution comparisons were assessed using the algorithms of Raymond and Rousset (Raymond, M. and Rousset, F., 1995. Evolution, 49, 1280-1283). Single locus Hardy-Weinberg analyses were performed based on Weir's algorithm (Weir, B., 1996. Genetic Data analysis II, Sinauer Associates, Sunderland, Mass.), using Genetic Data Analysis (GDA) Version 1.0 (d12) by P. O. Lewis and D. Zaykin, a free program distributed by the authors at the following URL: lewis.eeb.uconn.edu/lewishome/gda.html.

Prior to analyses, distributions for BMI, weight, body fat-mass, waist circumference and abdominal adipose tissue (assessed by computed tomography) were tested for skewness and kurtosis in the different study samples. All these variables showed skewness and kurtosis values below 1 and 4, respectively. Mean anthropometric values were compared between genotype classes using Student's t test. Analysis of covariance was used to adjust anthropometric variables for age. Logistic regression analyses were done on the dependent variable obesity defined as BMI greater than or equal to 30 for obese vs. BMI<30 for non-obese. Odds ratios were adjusted for the confounding effects of age and gender. All statistical analyses were performed with the SAS statistical package (SAS Institute, Cary, N.C.). The quantitative transmission disequilibrium tests (QTDT) were performed with the software “QTDT” version 2.2.1, (available at the following URL: well.ox.ac.uk/asthma/QTDT/download/index.html) using an orthogonal association model (Abecasis, G. et al., 2000. Am. J. Hum. Genet., 66:279-292).

### EXAMPLE 2

### Results

A thorough examination of the resistin gene locus for coding and non-coding variants was initiated.

In the polymorphism discovery phase of this examination, all four exons, the exon-intron boundaries, and 472 bp of the 5′ flanking region of the resistin gene were resequenced in 47 individuals from two study populations (Vohl M-C. et al., 2000. J. Lipid Res., 41:945-952; Vohl M-C. et al., 1999. Int. J. Obes. Relat. Meta. Disord., 23:918-925) representing a sample of diabetic, obese, and control individuals. In this phase of the study, seven variants (i.e., polymorphisms) were found: one substitution in the 3′ UTR (c.*62G>A), two SNPs in intron 2 (IVS2+39C>T and IVS2+181G>A), two SNPs in intron 3 (IVS3+30C>T and IVS3−16C>G) and two SNPs in the 5′ flanking region (g.-537A>C and g.-420C>G) (FIG. 1). Two of these polymorphisms have been previously described (c.*62G>A and IVS2+39C>T; Cao, H. and Hegele, R. 2001. J. Hum. Genet., 46:553-5) and one (g.-420C>G) had been identified by The SNP Consortium and is in GenBank's dbSNP (Reference SNP Id: 1862513). Since the two promoter polymorphisms are very close to each other, they were genotyped by sequencing genomic DNA. Two more rare 5′ flanking SNPs were identified in subsequent samples (g.-638G>A and g.-358G>A) (FIG. 1). These two SNPs appear to be in perfect LD, with the minor alleles always present in the same 11 heterozygotes among 590 Quebec individuals.

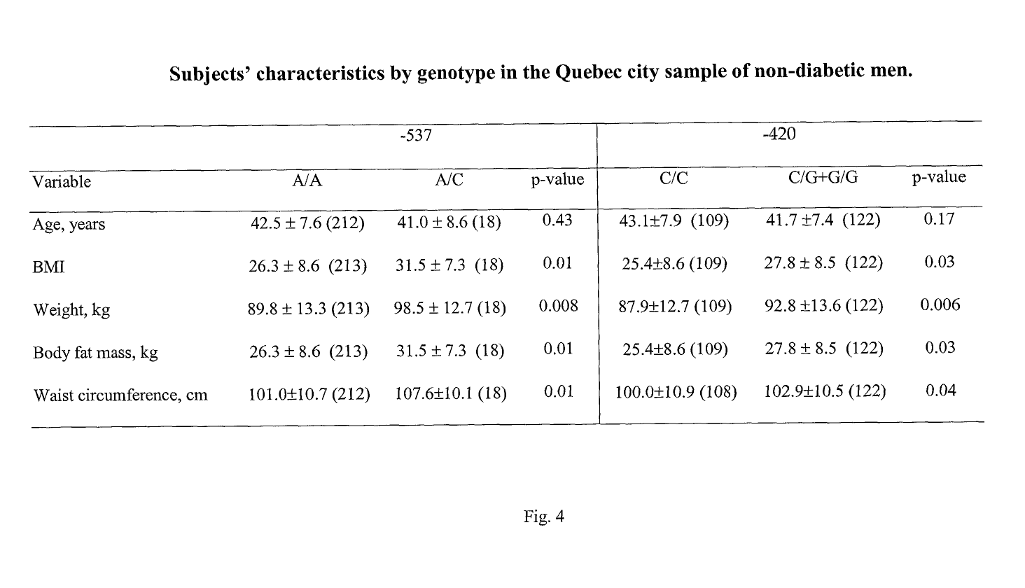

An association study was initiated in order to examine the role of the two more common 5′ flanking SNPs (g.-537A>C and g.-420C>G) in a type II diabetes case/control sample from the SLSJ region of Quebec and a population sample of men from the Quebec City area. Genotype distributions for these two variants did not differ significantly from Hardy-Weinberg equilibrium in either the Quebec City sample or the SLSJ case/controls. In addition, allelic frequencies of the two study samples were not significantly different from each other. In order to assess the contribution of the g.-537A>C and g.-420C>G polymorphisms to the development of type 2 diabetes, allele frequencies of these variants in the case/control study sample of diabetics and non-diabetics recruited from the SLSJ area were compared. As shown in FIG. 4, no difference in allele frequencies was observed between type 2 diabetics and non-diabetics. In a logistic regression model taking into account age and gender, neither the resistin g.-537A>C polymorphism nor the g.-420C>G polymorphism were significant contributors to diabetes.

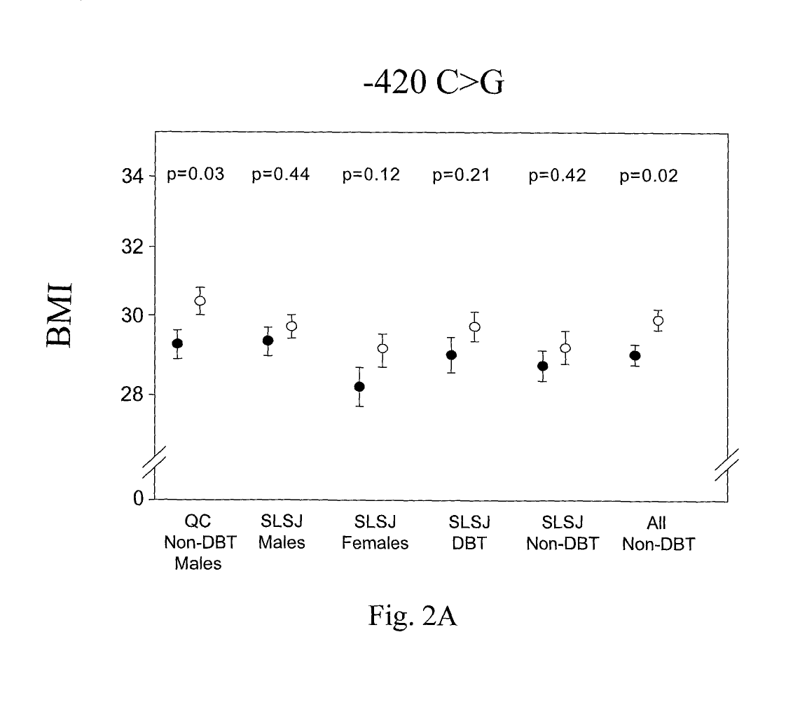

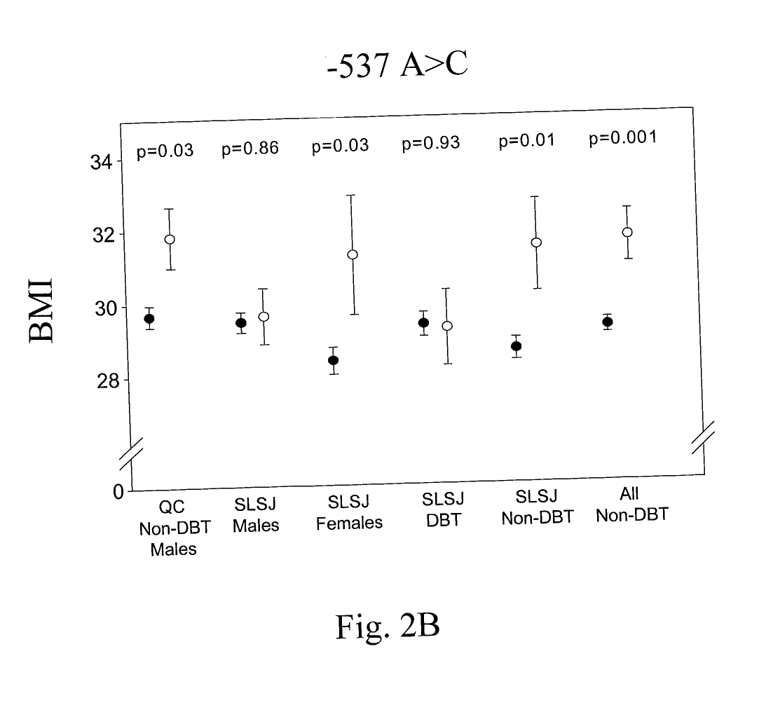

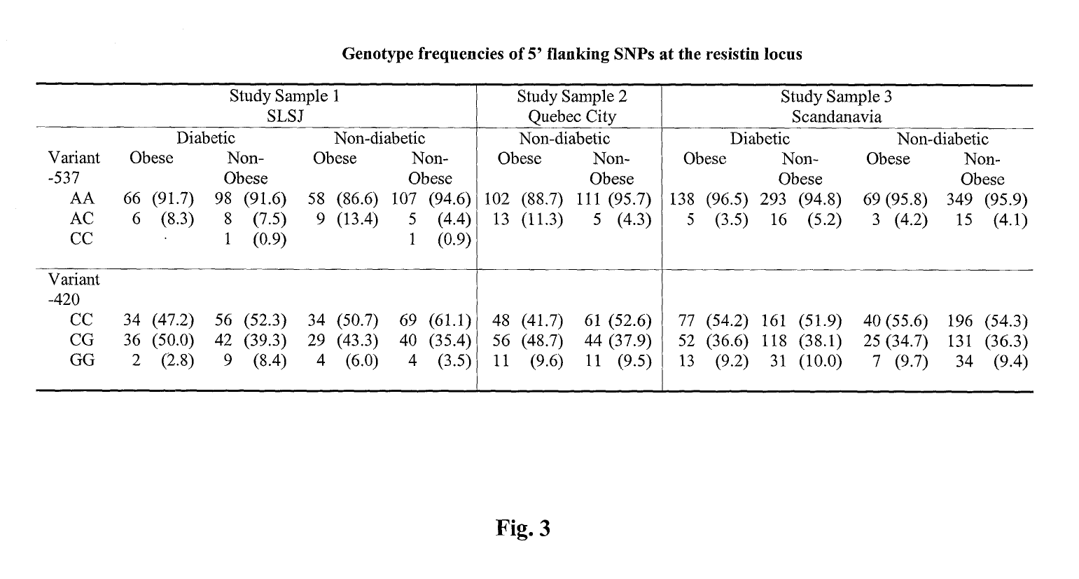

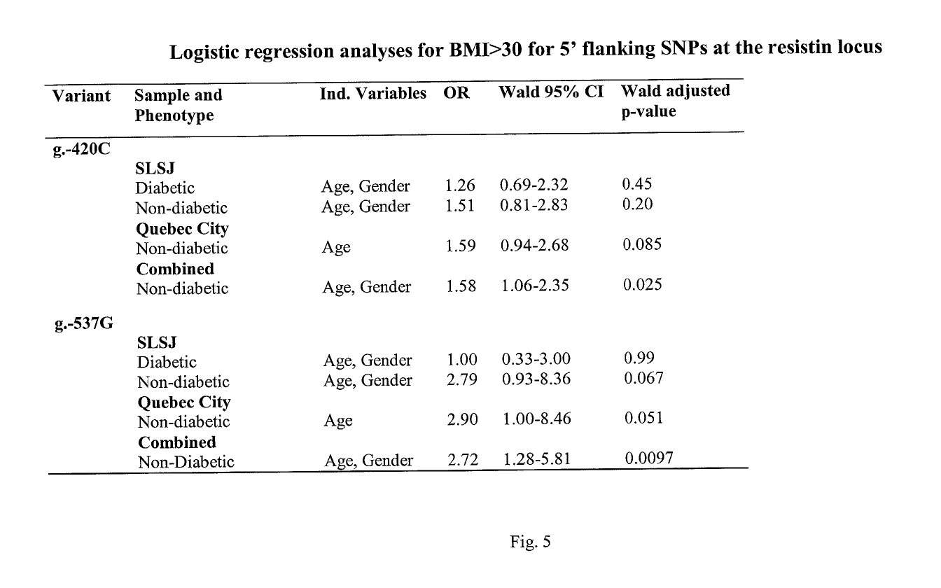

In the Quebec City sample, an increase in BMI (30.4 vs. 29.2 kg/m2, p=0.03) is associated with the presence of the g.-420 G allele compared to the C/C genotype (FIG. 2A). Similarly, an association was found with the presence of the g.-537 C allele compared to the A/A genotype for BMI (31.8 vs. 29.7 kg/m2), which is also significant (p=0.03) (FIG. 2B), despite the low frequency (3.9%) of this allele. In addition, several indices of obesity are significantly associated with the G allele at g.-420: weight (92.8 vs. 87.9 kg, p=0.006), body fat mass (27.8 vs. 25.4 kg, p=0.03), and waist circumference (102.9 vs. 100.0 cm, p=0.04) (FIG. 5). All of these parameters are affected by the C allele at position g.-537. All differences are statistically significant after adjustment for age.

Because an association of the g.-537 and c.-420 alleles with BMI was observed in the Quebec City sample, these alleles were further analyzed in the SLSJ diabetic and non-diabetic subjects for their association with BMI. No association was observed between either of the polymorphisms and BMI when the whole study population was included, but there was a statistically significant effect for carriers of the g.-537 C allele when only females were examined (p=0.03) (FIG. 2B). Waist circumference was also significantly higher in these women (90.9 vs. 86.4 cm, p=0.01). BMI was significantly higher in carriers of the g.-537 C (p=0.01) when only the non-diabetic subgroup of the SLSJ study population was examined (FIG. 2B). The most significant association of BMI and genotype was observed when all non-diabetics from both studies were combined, with g.-420G and g.-537C both being significant at p=0.017 and p=0.001, respectively (FIGS. 2A and B). All these differences remain significant after adjustment for age. In addition, in a family-based population sample from the SLSJ that had some overlap with the case/control sample, a QTDT showed a significant contribution of the g.-537C allele to a higher BMI when age and sex were included in the model (p=0.0386). As the SLSJ case/control study sample, this does not constitute an independent replication.

Using logistic regression analyses, odds ratios were calculated for a BMI greater than 30 kg/m2. There was a strong agreement between the odds ratios (OR) for the Quebec City and the SLSJ study samples when only non-diabetic individuals were examined. For both of these groups the OR was >1.5 for the g.-420G variant, and >2.7 for the g.-537C variant (FIG. 6). Finally, for all non-diabetics combined, the odds ratios for the G allele at g.-420C>G and the C allele at g.-537A>C are 1.58 (CI: 1.06-2.35, p=0.025) and 2.72 (CI: 1.28-5.81, p=0.01), respectively, when age and gender are included in the model (FIG. 6).

The g.-420 G and the g.-537 C alleles are in significant linkage disequilibrium with each other. In the 590 Quebec individuals studied so far, the C allele at position g.-537 is present only in individuals bearing the G allele at position g.-420. The average BMI of those non-diabetic individuals possessing the C allele at position g.-537 (and thus possessing both variant alleles) is higher than those possessing only the G allele at position g.-420 in (p=0.006).

It is possible that either or both of these two 5′ polymorphisms represent a base change in a transcription factor binding site affecting mRNA levels of resistin. A search for potential binding sites at positions g.-420 and g.-537 with MatInspector V2.2 (Quandt, K. et al., 1995. Nucl. Acids Res., 23:4878-4884) reveals several transcription factor motifs altered by these variant alleles (e.g., an API site is destroyed by the g.-537A>C).

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for determining an individual's susceptibility to obesity, comprising detecting in a biological sample obtained from the individual an allele at a polymorphic site genetically linked to the resistin gene locus, wherein the allele is further linked to obesity, and wherein detection of the allele is indicative of the patient's susceptibility to obesity.

2. The method of claim 1, wherein the allele is indicative of the individual having an increased probability of being susceptible to obesity.

3. The method of claim 1, wherein the allele is indicative of the individual having a decreased probability of being susceptible to obesity.

4. The method of claim 1, wherein susceptibility to obesity indicates a risk for an obesity-associated health disorder.

5. The method of claim 4, wherein the obesity-associated health disorder is selected from the group consisting of: diabetes mellitus, coronary heart disease, obesity, cancer and sleep-breathing disorders.

6. The method of claim 1, wherein the method of detecting the allele comprises a hybridization assay.

7. The method of claim 6, wherein the hybridization assay comprises use of a microarray.

8. A method for determining an individual's susceptibility to obesity, comprising detecting in a biological sample obtained from the individual an allele at a polymorphic site selected from the group consisting of: g.-638, g.-537, g.-420, g.-358, IVS2+39, IVS2+181, IVS3+30, IVS3−16, c.*62, and combinations thereof, and wherein detection of the allele is indicative of the individual's susceptibility to obesity.

9. The method of claim 8, wherein the individual is of French-Canadian descent.

10. The method of claim 8, wherein the polymorphic site is g.-537.

11. The method of claim 8, wherein the polymorphic site is g.-420.

12. The method of claim 8, wherein the allele detected is selected from the group consisting of: g.-638G>A, g.-537A>C, g.-420C>G, g.-358G>A, IVS2+39C>T, IVS2+181G>A, IVS3+30C>T, IVS3−16C>G, and c.62*G>A.

13. The method of claim 12, wherein the allele detected is g.-537A>C.

14. The method of claim 12, wherein the allele detected is g.-420C>G.

15. The method of claim 8, wherein the method of detecting the allele comprises a hybridization assay.

16. The method of claim 15, wherein the hybridization assay comprises use of a microarray.

17. A method for determining an individual's susceptibility to obesity or obesity-related health disorders, comprising
detecting in a biological sample obtained from the individual an allele at a polymorphic site in the coding sequence of the resistin gene, wherein the allele is further linked to obesity, and wherein detection of the allele is indicative of the individual's susceptibility to obesity.

18. The method of claim 17, wherein the individual is of French-Canadian descent.

19. The method of claim 17, wherein the method of detecting the allele comprises detecting the protein product of the resistin gene.

20. The method of claim 19, wherein the method of detecting the allele comprises the use of antibodies.

21. The method of claim 17, wherein the allele is c.62*G>A.

22. A method for determining an individual's predisposition to insulin resistance and health disorders associated with insulin resistance, comprising detecting a specific allele at a polymorphic site, wherein the allele is genetically linked to the resistin gene locus, and wherein detection of the allele is indicative of insulin resistance.

23. The method of claim 22, wherein the method of detecting the polymorphism comprises a hybridization assay.

24. The method of claim 23, wherein the hybridization assay comprises a microarray.

25. A method for determining an individual's predisposition to insulin resistance, comprising detecting a specific allele at a polymorphic site, wherein the allele is selected from the group consisting of: g.-638G>A, g.-537A>C, g.-420C>G, g.-358G>A, IVS2+39C>T, IVS2+181G>A, IVS3+30C>T, IVS3−16C>G, and c.62*G>A, and wherein detection of the allele is indicative of the individual's susceptibility to insulin resistance.

26. The method of claim 25, wherein the individual is of French-Canadian descent.

27. The method of claim 25, wherein the allele detected is g.-537A>C.

28. The method of claim 25, wherein the allele detected is g.-420C>G.

29. The method of claim 25, wherein the method of detecting the polymorphism comprises a hybridization assay.

30. The method of claim 29, wherein the hybridization assay comprises a microarray.

31. A method for determining an individual's predisposition to a health disorder associated with insulin resistance, comprising detecting a specific allele at a polymorphic site, wherein the allele is genetically linked to the resistin gene locus, and wherein detection of the allele is indicative of the health disorder associated with insulin resistance.

32. The method of claim 31, wherein the method of detecting the allele comprises a hybridization assay.

33. The method of claim 32, wherein the hybridization assay comprises a microarray.

34. A method for determining an individual's predisposition to a health disorder associated with insulin resistance, comprising detecting a specific allele at a polymorphic site, wherein the allele is selected from the group consisting of: g.-638G>A, g.-537A>C, g.-420C>G, g.-358G>A, IVS2+39C>T, IVS2+181G>A, IVS3+30C>T, IVS3−16C>G and c.62*G>A, and wherein detection of the allele is indicative of the individual's susceptibility to a health disorder associated with insulin resistance.

35. The method of claim 34, wherein the individual is of French-Canadian descent.

36. The method of claim 34, wherein the allele detected is g.-537A>C.

37. The method of claim 34, wherein the allele detected is g.-420C>G.

38. The method of claim 34, wherein the method of detecting the allele comprises a hybridization assay.

39. The method of claim 38, wherein the hybridization assay comprises a microarray.

40. The method of claim 34, wherein the health disorder is selected from the group consisting of: diabetes mellitus, coronary heart disease, obesity, cancer and sleep-breathing disorders.

41. A method for determining an individual's predisposition to type II diabetes, comprising detecting a specific allele at a polymorphic site, wherein the allele is genetically linked to the resistin gene locus, and wherein detection of the allele is indicative of type II diabetes.

42. The method of claim 41, wherein the method of detecting the allele comprises a hybridization assay.

43. The method of claim 42, wherein the hybridization assay comprises a microarray.

44. A method for determining an individual's predisposition to type II diabetes, comprising detecting a specific allele at a polymorphic site, wherein the allele is selected from the group consisting of: g.-638G>A, g.-537A>C, g.-420C>G, g.-358G>A, IVS2+39C>T, IVS2+181G>A, IVS3+30C>T, IVS3−16C>G, and c.62*G>A, and wherein detection of the allele is indicative of the individual's susceptibility to type II diabetes.

45. The method of claim 44, wherein the individual is of French-Canadian descent.

46. The method of claim 44, wherein the allele detected is g.-537A>C.

47. The method of claim 44, wherein the allele detected is g.-420C>G.

48. The method of claim 44, wherein the method of detecting the allele comprises a hybridization assay.

49. The method of claim 48, wherein the hybridization assay comprises a microarray.

50. A nucleic acid molecule comprising a nucleotide sequence from the resistin gene comprising an allele selected from the group consisting of: g.-638G>A, g.-537A>C, g.-420C>G, g.-358G>A, IVS2+181G>A, IVS3+30C>T and IVS3−16C>G, wherein the sequence flanks the allele on one or both sides.
